(19) United States
(12) Patent Application Publication
(10) Pub. No.: US 2008/0009641 A1
(43) Pub. Date: Jan. 10, 2008

(54) CHECKER FOR AQUEOUS COMPOSITION FOR STERILIZATION

(75) Inventor: Hideo MIYAZAKI, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/769,969

(22) Filed: Jun. 28, 2007

(30) Foreign Application Priority Data
Jul. 4, 2006 (JP) 2006-184006

(51) Int. Cl. C07C 409/24; C07D 307/12

(52) U.S. Cl. 549/486; 549/501; 562/2

(57) ABSTRACT

A checker which comprises potassium iodide, thiosulfate ion, and starch, wherein a weight ratio of the potassium iodide and the thiosulfate ion (potassium iodide/thiosulfate ion) is 0.70 to 0.3; and a kit comprising two or more of the aforementioned checkers, wherein the aforementioned weight ratio is different between said checkers are provided. The checker or the kit easily and precisely determines whether or not the concentration of an organic carboxylic acid peroxide such as peracetic acid in an aqueous composition for sterilization comprising the organic carboxylic acid peroxide as an active ingredient is an effective concentration.

### CROSS-REFERENCE TO RELATED APPLICATIONS

This application claims benefit of priority under 35 USC 119 to Japanese Patent Application No. 2006-184006 filed on Jul. 4, 2006, the disclosure of which is expressly incorporated herein by reference in its entirety.

### BACKGROUND OF THE INVENTION

1. Field of the invention

The present invention relates to a checker for an aqueous composition for sterilization. More specifically, the present invention relates to a checker which determines whether or not a concentration of an organic carboxylic acid peroxide in an aqueous composition for sterilization comprising the organic carboxylic acid peroxide as an active ingredient is an effective concentration, and a kit comprising the checker.

2. Description of the Prior Art

A medical apparatus, which is not disposable, is directly contacted with patients and therefore needs a safer sterilization for use in subsequent examinations and the like. As a composition for cleaning and sterilizing a medical apparatus such as endoscope, a composition containing an aldehyde such as glutaraldehyde as an active ingredient is conventionally known. However, an aldehyde such as glutaraldehyde has mutagenicity and a problem of weak sterilizing effect to spores. Therefore, a composition for sterilization which contains peracetic acid as an active ingredient has become widely used instead.

As the composition for sterilization containing peracetic acid as an active ingredient, Acecide (a registered trademark) is known as a commercial product (Acecide 6% disinfectant catalog; Saraya Co., Ltd., October 2001, the disclosure of which is expressly incorporated herein by reference in its entirety). Acecide consists of a base agent comprising peracetic acid, hydrogen peroxide, and acetic acid and a buffer agent. For a use for sterilization, purified water is added to the above agents to prepare an aqueous solution for use. The solution can be used repeatedly as a sterilizing agent for about 7 days. However, once the aqueous solution is prepared, temperature, ultraviolet ray, or incorporation of impurities lowers the concentration of peracetic acid. Therefore, it should be verified whether or not the solution has sterilization activity of baseline or a higher level, before use. The verification has been conducted by using a special test paper which changes color by the iodo-starch reaction to determine whether or not the above solution contains the effective concentration of peracetic acid. When the change of the color of the test paper becomes insufficient, peracetic acid in the above aqueous solution is determined to be below the effective concentration.

However, the change of color needs to be observed within 10 seconds after soaking of the test paper in the above aqueous solution. When time passes, the test paper exhibits sufficient change of color even in the solution which contains peracetic acid below the effective concentration. Therefore, there have been problems of a restricted determination procedure and high risk of erroneous determination.

### SUMMARY OF THE INVENTION

The present invention was conducted in view of the above problems, and an object of the present invention is to provide a mean of easily and precisely determining whether or not a concentration of an organic carboxylic acid peroxide such as peracetic acid in an aqueous composition for sterilization comprising the organic carboxylic acid peroxide as an active ingredient is an effective concentration.

In order to achieve the foregoing object, the inventor of the present invention conducted intensive researches. As a result, he found that preservation of the change of color is influenced by a weight ratio of potassium iodide and thiosulfate ion which is added as an aiding agent, in the test paper using the reaction wherein iodide ion changed to iodine by the oxidizing action of a peracid, which is then react with starch to exhibit change of color to blue purple. The present invention was achieved on the basis of this finding.

The present invention thus provides a checker of determining whether or not a concentration of an organic carboxylic acid peroxide in an aqueous composition for sterilization comprising the organic carboxylic acid peroxide as an active ingredient is an effective concentration, which comprises potassium iodide, thiosulfate ion, and starch, wherein a weight ratio of the potassium iodide and the thiosulfate ion (potassium iodide/thiosulfate ion) is 0.70 to 0.3.

As a preferred embodiment of the present invention, provided is the aforementioned checker, wherein a weight ratio of the potassium iodide and the starch (potassium iodide/starch) is 0.3 to 0.1.

As further preferred embodiments of the present invention, provided are the aforementioned checker, wherein the carboxylic acid peroxide is peracetic acid; the aforementioned checker, wherein the carboxylic acid peroxide is a peroxycarboxylic acid represented by the general formula (I):

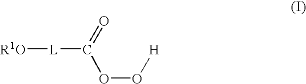

wherein R¹ represents a substituted or unsubstituted C1-C10 alkyl group, or a substituted or unsubstituted C6-C10 aryl group, L represents a substituted or unsubstituted C2-C12 divalent linking group, or R¹ and L may combine to form a ring; the aforementioned checker, wherein the carboxylic acid peroxide is a peroxycarboxylic acid represented by the general formula (II) or (III):

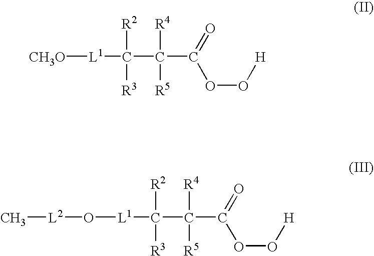

wherein R², R³, R⁴, and R⁵ each independently represents hydrogen, a C1-C4 alkyl group, a C1-C4 alkoxy group, or a C1-C4 alkoxy-substituted C1-C4 alkyl group, L² represents a substituted or unsubstituted C1-C9 alkylene group, or a substituted or unsubstituted C6-C9 arylene group, and L¹ represents a substituted or unsubstituted C1-C10 divalent linking group; and the aforementioned checker, wherein the carboxylic acid peroxide is a peroxycarboxylic acid represented by the general formula (IV) or (V):

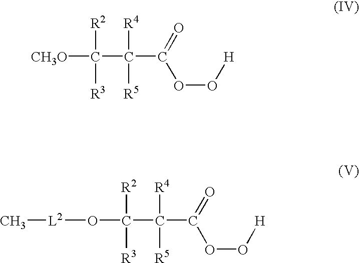

wherein R², R³, R⁴, and R⁵ each independently represents hydrogen, a C1-C4 alkyl group, a C1-C4 alkoxy group, or a C1-C4 alkoxy-substituted C1-C4 alkyl group, L² represents a substituted or unsubstituted C1-C9 alkylene group, or a substituted or unsubstituted C6-C9 arylene group.

As further preferred embodiments of the present invention, provided are the aforementioned checker which comprises a paper, a non-woven fabric, or a porous polymer sheet as a base material; and the aforementioned checker, wherein the effective concentration is 27 mM or more.

From another aspect of the present invention, provided are a kit of determining whether or not a concentration of an organic carboxylic acid peroxide in an aqueous composition for sterilization comprising the organic carboxylic acid peroxide as an active ingredient is an effective concentration, which comprises two or more of the aforementioned checkers, wherein the weight ratio of the potassium iodide and the thiosulfate ion is different between said checkers; a kit of determining whether or not a concentration of an organic carboxylic acid peroxide in an aqueous composition for sterilization comprising the organic carboxylic acid peroxide as an active ingredient is an effective concentration, which comprises three of the aforementioned checkers, wherein the weight ratio of the potassium iodide and the thiosulfate ion is different between said checkers.

From further aspect of the present invention, provided are a method of determining whether or not a concentration of an organic carboxylic acid peroxide in an aqueous composition for sterilization comprising the organic carboxylic acid peroxide as an active ingredient is an effective concentration, by using a color-change reaction of a checker comprising potassium iodide, thiosulfate ion, and starch, wherein two or more of the checkers having a different weight ratio of the potassium iodide and the thiosulfate ion to each other are used in combination.

### DESCRIPTION OF THE PREFERRED EMBODIMENTS

Iodo-starch reaction is a reaction wherein starch and iodine forms a complex to exhibit blue purple color. In the presence of potassium iodide and starch, iodide ion changed to iodine by the oxidizing action of a peracid to exhibit the above change of color, the reaction thus can be used for determination as to whether peracid is present or not. By setting the concentration of potassium iodide and starch so that the change of color can be distinguished at the critical point between effective and ineffective concentrations of an organic carboxylic acid peroxide in an aqueous composition for sterilization comprising the acid peroxide as an active ingredient, the reaction can be used for determination as to whether the concentration of the organic carboxylic acid peroxide is an effective concentration or not.

The checker of the present invention is a checker using the iodo-starch reaction as described above, and is characterized in that the weight ratio of potassium iodide and thiosulfate ion which is added as a reducing agent and an agent for aiding preservation of the color (potassium iodide/ thiosulfate ion) is 0.70 to 0.3. The weight ratio is preferably 0.60 to 0.3, more preferably 0.40 to 0.3. In the specification, ranges indicated with “to” mean ranges including the numerical values before and after “to” as the minimum and maximum values.

According to the analysis conducted by the present inventor, the weight ratio of potassium iodide and thiosulfate ion in the conventionally known test paper was 0.75. By preparing the test paper having the weight ratio of potassium iodide and thiosulfate ion of 0.7 or less in the present invention, retention time of the color or the like was substantially improved. Further, the weight ratio of potassium iodide and thiosulfate ion is preferably 0.3 or more in order to obtain a sufficient change of color in the presence of an organic carboxylic acid peroxide at an effective concentration.

The above weight ratio can be controlled in a solution for preparing the checker.

In the solution for preparing the checker, water, for example, is used as solvent, and potassium iodide, starch, and thiosulfate ion is dissolved.

Potassium iodide may be a commercial product for a reagent or a material for manufacturing. For example, “Potassium iodide” (Wako Pure Chemical Industries, Ltd. 164-03977) can be used. The concentration of potassium iodide in the solution for preparing the checker may be in the range of 0.1 to 10 weight %.

Starch may be derived from corn, potato or the like, and preferably be susceptible to be solubilized. For example, “Soluble starch” (Wako Pure Chemical Industries, Ltd. 197-03987) can be used. The concentration of starch in the solution for preparing the checker may be in the range of 0.5 to 10 weight %. The weight ratio of potassium iodide and the above starch (potassium iodide/starch) is preferably 0.3 to 0.1.

As the resource of the thiosulfate ion, a thiosulfate such as sodium thiosulfate, potassium thiosulfate, calcium thiosulfate, or ammonium thiosulfate may be used. Ammonium thiosulfate may preferably be used. The concentration of thiosulfate ion in the solution for preparing the checker is adjusted so that the concentration ratio with potassium iodide can be in the aforementioned range.

The solution for preparing the checker may be further added with sodium sulfite for the purpose of preventing oxidative decomposition, potassium chloride for the purpose of controlling ion concentration, and an antibacterial agent for the purpose of preventing deterioration caused by environmental microorganisms.

The checker of the present invention may be prepared by soaking a base material in the solution for preparing the checker and by drying the material. As the base material, a paper, a non-woven fabric, a porous polymer sheet, or the like may be used.

The paper preferably has a high water absorption rate, and an example includes a filter paper for quantitative analysis, No.5C (ADVANTEC Toyo). As the non-woven fabric, material which is used for filter or the like can be used. An example includes “Supulitoppu SP” (Nippon non-woven fabrics Co., Ltd.) which is a polypropylene non-woven fabric. An example of the porous polymer sheet includes a cellulose-condensed ester membrane filter (MILLIPORE), which is a commercial product used for filtration.

Further, the checker of the present invention may include a material for conveniences of use.

An example of the above material includes a plastic plate. For example, a plastic plate of 0.1 mm to 0.5 mm thick, 5 cm to 10 cm length, and 3 mm to 2 cm width may be bonded with a base material containing potassium iodide and the like, to be provided as the checker.

By soaking the checker prepared as above in an aqueous composition for sterilization comprising an organic carboxylic acid peroxide as an active ingredient and by observing the change of color, it is possible to determine whether or not the aqueous composition contains the organic carboxylic acid peroxide at an effective concentration, i.e., it is possible to determine as an effective concentration when the checker exhibits a complete change of color to deep purple color; and as an ineffective concentration when the checker exhibits a incomplete change of color (such as unevenly changed color). The determination (observation) can be conducted 10 seconds after the above soaking as in the conventional method. However, a precise determination can also be conducted 20 seconds to 5 minutes, preferably 20 to 120 seconds after the above soaking.

For a more precise determination, two or more checkers of the present invention which have different weight ratio of potassium iodide and thiosulfate ion to each other can be also preferably used in combination. By using plural checkers, results which differ in the degree of the change of color and the preservation of the colored state can be obtained at the same time. Therefore, a precise determination becomes possible particularly in the vicinity of the effective concentration. As the weight ratio of potassium iodide and thiosulfate ion (potassium iodide/ thiosulfate ion), two values near the upper limit and the lower limit in the range of 0.70 to 0.3; three values near the upper limit, the lower limit, and an intermediate in the range of 0.70 to 0.3; or the like are preferably used. For example, the checkers each having the weight ratios of 0.70 to 0.6, 0.55 to 0.45, and 0.4 to 0.3 can be used in combination. Two or more checkers of the present invention which have different weight ratio of potassium iodide and thiosulfate ion to each other can be provided as a kit. As the kit, an example includes a kit wherein plastic plates bonded with a base material containing potassium iodide and the like are combined in a reed shape. However, the kit may be in any shape as long as the shape enables the color-change reactions on the plural checkers to start at the same time, and is not particularly limited.

The organic carboxylic acid peroxide of which the effective concentration can be determined by using the checker of the present invention is not particularly limited. Preferable examples include peracetic acid, and a peroxycarboxylic acid represented by the following general formulae (I) to (V):

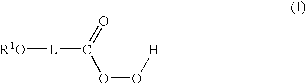

wherein R¹ represents a substituted or unsubstituted C1-C10 alkyl group, or a substituted or unsubstituted C6-C10 aryl group, L represents a substituted or unsubstituted C2-C12 divalent linking group, or R¹ and L may combine to form a ring;

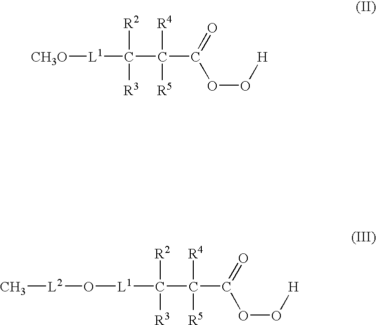

wherein R², R³, R⁴, and R⁵ each independently represents hydrogen, a C1-C4 alkyl group, a C1-C4 alkoxy group, or a C1-C4 alkoxy-substituted C1-C4 alkyl group, L² represents a substituted or unsubstituted C1-C9 alkylene group, or a substituted or unsubstituted C6-C9 arylene group, and L¹ represents a substituted or unsubstituted C1-C10 divalent linking group;

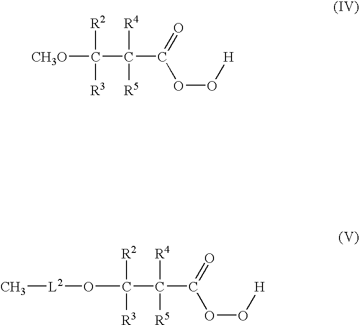

wherein R², R³, R⁴, and R⁵ each independently represents hydrogen, a C1-C4 alkyl group, a C1-C4 alkoxy group, or a C1-C4 alkoxy-substituted C1-C4 alkyl group, L² represents a substituted or unsubstituted C1-C9 alkylene group, or a substituted or unsubstituted C6-C9 arylene group.

As the unsubstituted C1-C10 alkyl group represented by R¹, examples include methyl group, ethyl group, propyl group, isopropyl group, butyl group, isobutyl group, sec-butyl group, tert-butyl group, pentyl group, isopentyl group, neopenty group, tert-penty group, hexyl group, heptyl group, octyl group, nonyl group, decyl group, 2-ethylhexyl group and the like. The C1-C10 alkyl group represented by R¹ may have a substituent,-which is preferred to be a substituent other than a substituent having OH group (hydroxy group, carboxy group, hydroperoxy group, hydroperoxycarbonyl group and the like). As the substituent of the C1-C10 alkyl group represented by R¹, examples include a halogen atom, an alkoxy group, and an alkylsulfonyl group.

As the unsubstituted C6-C10 aryl group represented by R¹, an example includes phenyl group. The C6-C10 aryl group represented by R¹ may have a substituent. Examples of the substituent include an alkyl group, an alkoxy group, sulfonyl group, hydroxy group, carboxyl group and the like.

As R¹, a substituted or unsubstituted C1-C4 alkyl group is preferred, and an unsubstituted C1-C4 alkyl group is more preferred. As the C1-C4 alkyl group represented by R¹, examples include methyl group, ethyl group, propyl group, isopropyl group, butyl group, isobutyl group, sec-butyl group, tert-butyl group and the like. As R¹, methyl group or ethyl group is most preferred. However, R¹ is not limited to these examples.

In the general formula (I), L represents a substituted or unsubstituted divalent linking group having 2 to 12 carbon atoms. As the substituent, examples include an alkyl group, an alkoxy group, hydroxy group, an alkoxy-substituted alkyl group, carboxyl group and the like. The linking group may have an atom other than carbon atom such as an oxygen atom or nitrogen atom in the group. As L, a preferable example includes CHR¹¹-L¹² (R¹¹ represents hydrogen atom or a substituted or unsubstituted C1-C4 alkyl group, and L¹² represents a substituted or unsubstituted C1-C11 divalent linking group). As L¹², a substituted or unsubstituted C1-C4 divalent linking group is preferred, a substituted or unsubstituted methylene group is more preferred, methylene group or methyl-substituted methylene group (CH(CH₃)) is further preferred, and methylene group is most preferred.

In the general formula (II), (III), or (V), L¹ represents a substituted or unsubstituted C1-C10 divalent linking group. As the substituent, examples include an alkyl group, an alkoxy group, hydroxy group, an alkoxy-substituted alkyl group, carboxyl group and the like. The linking group may have an atom other than carbon atom such as an oxygen atom or nitrogen atom in the group. As L¹, a preferable example includes CHR¹²-L¹³ (R¹² represents hydrogen atom or a substituted or unsubstituted C1-C4 alkyl group, and L¹³ represents a substituted or unsubstituted C1-C9 divalent linking group). As L¹³, a substituted or unsubstituted C1-C4 divalent linking group is preferred, a substituted or unsubstituted methylene group is more preferred, methylene group or methyl-substituted methylene group (CH(CH₃)) is further preferred, and methylene group is most preferred.

As the C1-C4 alkyl group represented by R², R³, R⁴, or R⁵, examples include methyl group, ethyl group, propyl group, isopropyl group, butyl group, isobutyl group, sec-butyl group, and tert-butyl group and a preferable example includes methyl group. However, the alkyl group is not limited to these examples. As the C1-C4 alkoxy group represented by R², R³, R⁴, or R⁵, examples include methoxy group, ethoxy group, propoxy group, and butoxy group, and preferable examples includes methoxy group and ethoxy group. However, the alkoxy group is not limited to these examples. As the C1-C4 alkoxy-substituted C1-C4 alkyl group represented by R², R³, R⁴, or R⁵, examples include methoxymethyl group ethoxymethyl group, propoxymethyl group, butoxymethyl group and ethoxyethyl group, and preferable examples includes methoxymethyl group and ethoxymethyl group However, the alkoxy-substituted alkyl group is not limited to these examples.

In the general formula (III) or (V), as the divalent linking group represented by L², a substituted or unsubstituted C1-C9 alkylene group is preferred, a substituted or unsubstituted C1-C3 alkylene group is more preferred, an unsubstituted C1-C3 alkylene group is further more preferred. As the unsubstituted C1-C3 alkylene group represented by L², examples include methylene group or ethylene group, and a preferable example includes methylene group.

The peroxycarboxylic acid represented by the above general formulae (I) to (V) may have one or more asymmetric carbons depending on the types of the substituents. The peroxycarboxylic acid may be any of the stereoisomers such as optically pure stereoisomers based on one or more asymmetric carbons, any mixtures of said stereoisomers, racemates, diastereoisomers based on two or more asymmetric carbons, any mixtures of said diastereoisomers and the like.

Specific examples of the aforementioned peroxycarboxylic acid are shown below. However, the scope of the present invention is not limited to the following compounds.

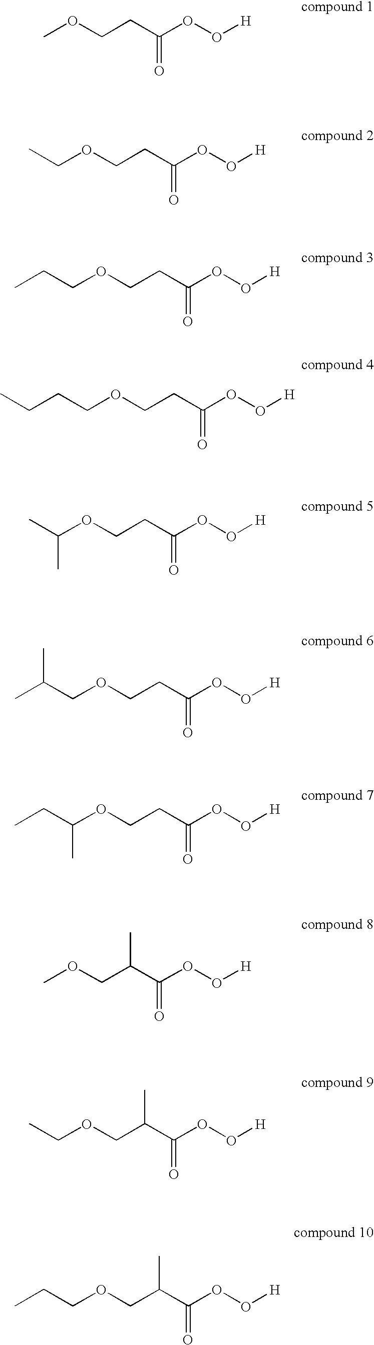

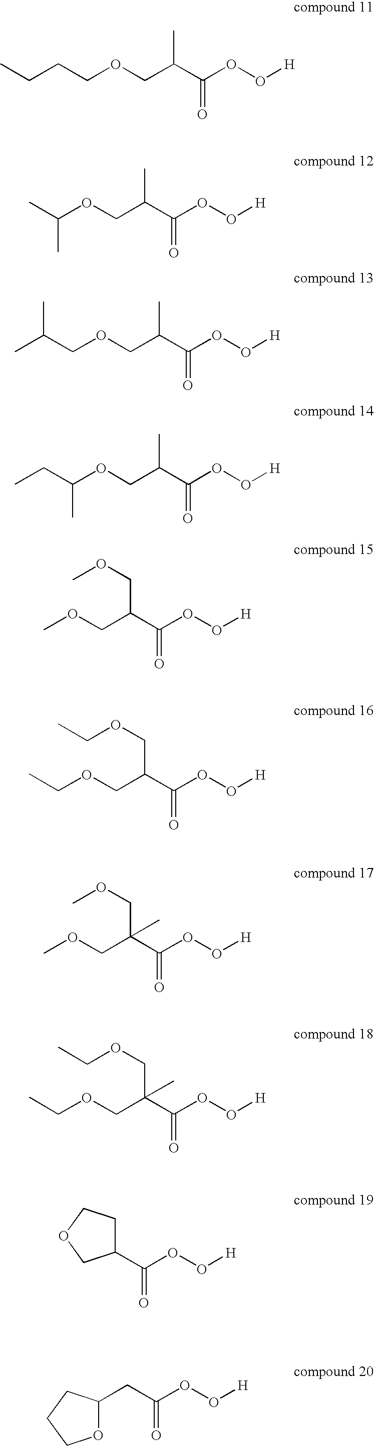

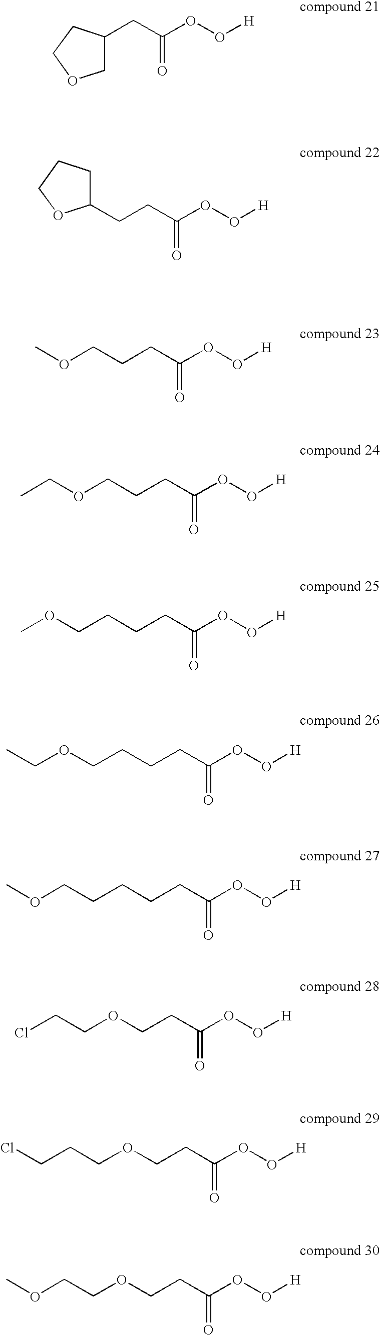

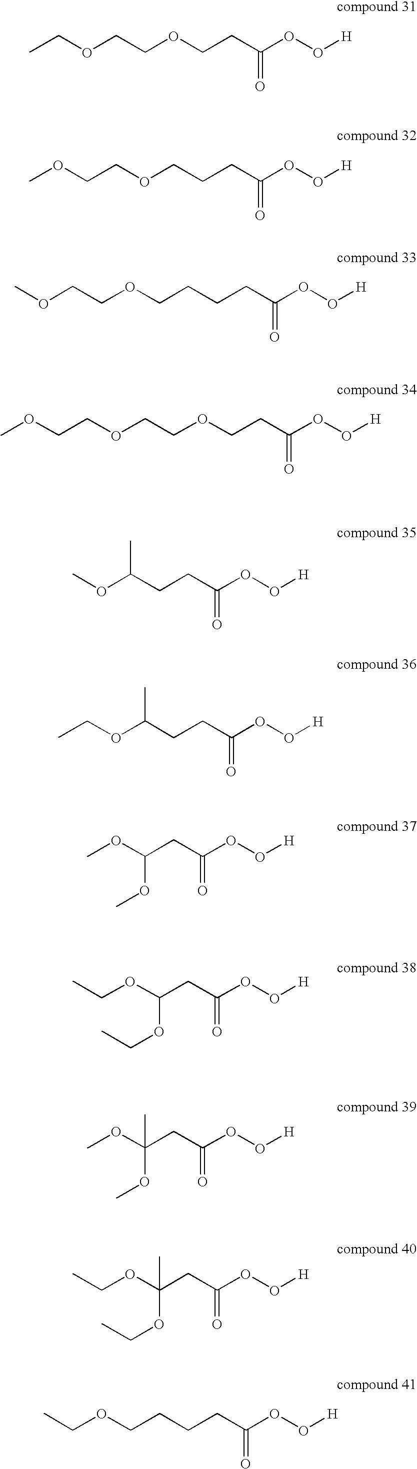

The peroxycarboxylic acid represented by the above general formulae (I) to (V) can be prepared according to known methods for preparing peroxycarboxylic acid such as that described in Organic peroxides, vol. I, pp 313-474 (Daniel Swern, John Wiley & Sons, Inc., New York, 1970), and the like. Specifically, a peroxycarboxylic acid can be prepared, for example, by a reaction of a carboxylic acid with hydrogen peroxide. In the reaction of a carboxylic acid with hydrogen peroxide, use of a strong acid is effective as a catalyst of the reaction. As the strong acid, a strongly acidic ion-exchange resin having sulfonic acid group and a solid acid catalyst such as Nafion, as well as sulfuric acid, phosphoric acid or the like can be used.

As a carboxylic acid used in the above preparation of a peroxycarboxylic acid, a carboxylic acid suitably prepared by a known method, as well as a commercially available carboxylic acid, can be used. For example, 3-alkoxypropionic acid can be prepared by a reaction of β-propiolactone and an alcohol as described in J. Am. Chem. Soc., 70, 1004 (1948) or by a hydrolysis of a 3-alkoxypropionic acid ester obtained from a reaction of an acrylic acid ester and an alkoxide as described in J. Am. Chem. Soc., 69, 2967(1947), ibid., 77, 754 (1955).

A carboxylic acid which is alkoxy-substituted at a position further than 3-position from a carbonyl group can be prepared by a reaction of a β-δ lactone and an alcohol as described in J. Org. Chem., 59, 2253(1994).

The aqueous composition for sterilization which can be determined of the effective concentration by the checker of the present invention comprises, for example, one or more of peracetic acid and the aforementioned peroxycarboxylic acid represented by the above general formulae (I) to (V). In the present specification, “an aqueous composition” means a composition in a state of solution comprising water.

The effective concentration of the organic carboxylic acid peroxide in the aqueous composition for sterilization, which is slightly different depending on the types of the peroxide, is generally 27 mM or more. The effective concentration may be determined by testing sterilization activity of the aqueous composition for sterilization in relation to concentrations of the organic carboxylic acid peroxide used, if necessary.

The aqueous composition for sterilization may comprise, other than peracetic acid or the aforementioned peroxycarboxylic acid, acetic acid or a carboxylic acid corresponding to the peroxycarboxylic acids, and/or hydrogen peroxide. In the present specification, “a carboxylic acid corresponding to a peroxycarboxylic acid” means a compound which has carboxyl group instead of peroxycarboxylic group of the peroxycarboxylic acid.

The aqueous composition for sterilization may comprise one or more additives other than the aforementioned ingredients. Examples of the additives includes corrosion inhibitors, solubilizing agents, pH regulators, metal chelating agents, stabilizing agents, surfactants, antiredeposition agents and the like.

The term “sterilization” used in the present specification includes meanings of “cleaning”, “disinfection”, “antibacterial function”, and “complete sterilization” and the like. Use of the aqueous composition for sterilization is not particularly limited. The aqueous composition for sterilization may be used for sterilization of a polluted vapor phase, as well as a liquid and a solid. When the aqueous composition for sterilization is used for sterilization of a medical apparatus such as endoscope, sterilization may be conducted by soaking the apparatus in the aqueous composition to achieve a contact of the apparatus with the composition. The aqueous composition for sterilization can be repeatedly used. However, the concentration of the organic carboxylic acid peroxide in the composition decreases by being used plural times or with passage of time. By determining whether or not the organic carboxylic acid peroxide is contained in the composition before use at an effective concentration with the checker of the present invention, the aqueous composition for sterilization can be efficiently used.

### EXAMPLES

The present invention will be explained more specifically with reference to examples. However, the scope of the present invention is not limited to the following examples.

### Example 1

### Preparation of a Checker of a Concentration of an Organic Carboxylic Acid Peroxide

(Preparation of a solution of starch)

| | | |
|---|---|---|
| Purified water | 800 | ml |
| Soluble starch | 30 | g (manufactured by Wako Pure Chemical |
| | | Industries, Ltd.) |
| KCl | 0.88 | g (manufactured by Wako Pure Chemical |
| | | Industries, Ltd.) |
| Sodium sulfite | 1.18 | g (manufactured by Wako Pure Chemical |
| | | Industries, Ltd.) |
| Ammonium thiosulfate | 14.85 | g (manufactured by Wako Pure Chemical |
| | | Industries, Ltd.) |

The starch was put in a 2L beaker and allowed to swell for about 30 minutes by gradual addition of purified water. Then, the beaker was placed in warm water at about 50° C., and the starch was dissolved by stirring. Each of KCl, sodium sulfite, and ammonium thiosulfate was added to the starch solution, and dissolved. Then the solution was cooled to 30° C. or lower, added with purified water until the amount of the solution reached to 1L.

### (Preparation of Potassium Iodide Starch Solution)

The above-prepared starch solution was divided into 300 mL portions to obtain fractions A, B, and C. The following amount of KI was added to each of the fractions and dissolved.

A: 2.22 g; B: 1.79 g; C: 1.31 g

### (Preparation of a Checker)

A filter paper of about 12 cm² (No. 5C manufactured by ADVANTEC Toyo) was soaked in each of the above-prepared potassium iodide starch solution filled in a dish-shaped container. After drying in a cool and dark space, each of the filter papers was cut to 6 mm square, and bonded by using two-sided tape to the edge of a white plastic plate (a mount for crystal paper: manufactured by Fuji Photo Film) which has width of 6 mm and length of 7 cm, to obtain a checker.

The above-prepared checkers were named as Checker A, Checker B, and Checker C, respectively. Weight ratio of potassium iodide and thiosulfate ion (KI/S₂O₃) was calculated for each of the Checker A to C, to obtain the following values.

A: 0.66; B: 0.53; C: 0.39

### (Preparation of a Comparative Checker)

A comparative checker D was prepared in a similar manner to the above preparation, except that the preparation was conducted so that the KI/S₂O₃ value of the comparative checker was 0.75.

### Example 2

### Verification of the Concentration of the Organic Carboxylic Acid Peroxide

Solutions of 40 mM (effective concentration) and 25 mM (ineffective concentration) were prepared for each of peracetic acid and the above example compound 2. The preparation method followed the method disclosed in WO2006-068306, the disclosure of which is expressly incorporated herein by reference in its entirety. The four solutions of 10 ml each were pored to Petri dishes. The Checkers A to D prepared in Example 1 were soaked in the above solutions for two seconds. The Checkers, which were removed from the solutions, were contacted with commercial paper towel at the edge to remove the residual liquid. Conditions of color-change of the Checkers were observed with time passage from the start of the soaking. The results are shown in Tables 1 to 4. The symbols in Tables represent the following conditions.

\textcircled{∘}: complete deep purple; ◯: purple; Δ: purple coloring but uneven; x: uneven brown

TABLE 1
Conditions of color-change of Checkers after 10 seconds

| Concentration | Checker | | | |
|---|---|---|---|---|
| of Solution | A | B | C | D |
| Peracetic acid 40 mM | ⊚ | ◯ | ◯ | ⊚ |
| Example compound 2 | ⊚ | ◯ | ◯ | ⊚ |
| 40 mM | | | | |
| Peracetic acid 25 mM | Δ | Δ | X | ◯ |
| Example compound 2 | Δ | Δ | X | ◯ |
| 25 mM | | | | |
| remarks | Present | Present | Present | Comparative |
| | invention | invention | invention | example |

TABLE 2
Conditions of color-change of Checkers after 20 seconds

| Concentration | Checker | | | |
|---|---|---|---|---|
| of Solution | A | B | C | D |
| Peracetic acid 40 mM | ⊚ | ⊚ | ◯ | ⊚ |
| Example compound 2 | ⊚ | ⊚ | ◯ | ⊚ |
| 40 mM | | | | |
| Peracetic acid 25 mM | ◯ | Δ | Δ | ⊚ |
| Example compound 2 | ◯ | Δ | Δ | ⊚ |
| 25 mM | | | | |
| remarks | Present | Present | Present | Comparative |
| | invention | invention | invention | example |

TABLE 3
Conditions of color-change of Checkers after 60 seconds

| Concentration | Checker | | | |
|---|---|---|---|---|
| of Solution | A | B | C | D |
| Peracetic acid 40 mM | ⊚ | ⊚ | ⊚ | ⊚ |
| Example compound 2 | ⊚ | ⊚ | ⊚ | ⊚ |
| 40 mM | | | | |
| Peracetic acid 25 mM | ◯ | Δ | Δ | ⊚ |
| Example compound 2 | ◯ | Δ | Δ | ⊚ |
| 25 mM | | | | |
| remarks | Present | Present | Present | Comparative |
| | invention | invention | invention | example |

TABLE 4
Conditions of color-change of Checkers after 120 seconds

| Concentration | Checker | | | |
|---|---|---|---|---|
| of Solution | A | B | C | D |
| Peracetic acid 40 mM | ⊚ | ⊚ | ⊚ | ⊚ |
| Example compound 2 | ⊚ | ⊚ | ⊚ | ⊚ |
| 40 mM | | | | |
| Peracetic acid 25 mM | ⊚ | ◯ | Δ | ⊚ |
| Example compound 2 | ⊚ | ◯ | Δ | ⊚ |
| 25 mM | | | | |
| remarks | Present | Present | Present | Comparative |
| | invention | invention | invention | example |

As is clear in Tables 1 to 4, determination as to whether the solution had a effective concentration or not was not possible after 20 seconds by using the checker of comparative example, whereas was possible by using the checker of the present invention, i.e., Checker A, Checker B, and Checker C distinguished the ineffective concentration more reliably. Further, the determination was possible even after 120 seconds by using the checker of the present invention which has weight ratio of potassium iodide and thiosulfate ion of 0.53 or 0.39. The gradual color-change conditions shown in Table 4 indicates that, by using two checkers which have different weight ratio of potassium iodide and thiosulfate ion to each other, highly precise determination can be achieved and the available determination time can be prolonged.

### INDUSTRIAL APPLICABILITY

The present invention provides a checker which can easily and precisely determine whether or not a concentration of an organic carboxylic acid peroxide such as peracetic acid in an aqueous composition for sterilization comprising the organic carboxylic acid peroxide as an active ingredient is an effective concentration.

1. A checker of determining whether or not a concentration of an organic carboxylic acid peroxide in an aqueous composition for sterilization comprising the organic carboxylic acid peroxide as an active ingredient is an effective concentration, which comprises potassium iodide, thiosulfate ion, and starch, wherein a weight ratio of the potassium iodide and the thiosulfate ion (potassium iodide/thiosulfate ion) is 0.70 to 0.3.

2. The checker according to claim 1, wherein a weight ratio of the potassium iodide and the starch (potassium iodide/starch) is 0.3 to 0.1.

3. The checker according to claim 1, wherein the organic carboxylic acid peroxide is peracetic acid.

4. The checker according to claim 1, wherein the organic carboxylic acid peroxide is a peroxycarboxylic acid represented by the general formula (I): 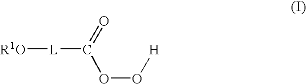
wherein R¹ represents a substituted or unsubstituted C1-C10 alkyl group, or a substituted or unsubstituted C6-C10 aryl group, L represents a substituted or unsubstituted C2-C12 divalent linking group, or R¹ and L may combine to form a ring.

5. The checker according to claim 1, wherein the organic carboxylic acid peroxide is a peroxycarboxylic acid represented by the general formula (II) or (III): 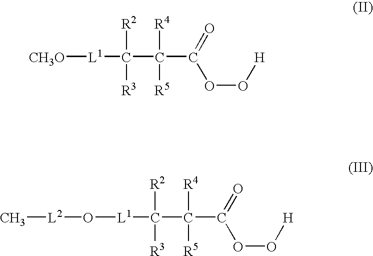
wherein R², R³, R⁴, and R⁵ each independently represents hydrogen, a C1-C4 alkyl group, a C1-C4 alkoxy group, or a C1-C4 alkoxy-substituted C1-C4 alkyl group, L² represents a substituted or unsubstituted C1-C9 alkylene group, or a substituted or unsubstituted C6-C9 arylene group, and L¹ represents a substituted or unsubstituted C1-C10 divalent linking group.

6. The checker according to claim 1, wherein the organic carboxylic acid peroxide is a peroxycarboxylic acid represented by the general formula (IV) or (V): 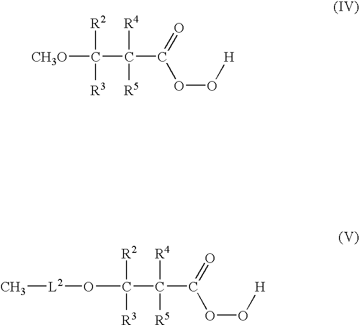
wherein R², R³, R⁴, and R⁵ each independently represents hydrogen, a C1-C4 alkyl group, a C1-C4 alkoxy group, or a C1-C4 alkoxy-substituted C1-C4 alkyl group, L² represents a substituted or unsubstituted C1-C9 alkylene group, or a substituted or unsubstituted C6-C9 arylene group.

7. The checker according to claim 1, which comprises a paper, a non-woven fabric, or a porous polymer sheet as a base material.

8. The checker according to claim 1, wherein the effective concentration is 27 mM or more.

9. A kit of determining whether or not a concentration of an organic carboxylic acid peroxide in an aqueous composition for sterilization comprising the organic carboxylic acid peroxide as an active ingredient is an effective concentration, which comprises two or more of the checkers according to claim 1, wherein the weight ratio of the potassium iodide and the thiosulfate ion is different between said checkers.

10. A kit of determining whether or not a concentration of an organic carboxylic acid peroxide in an aqueous composition for sterilization comprising the organic carboxylic acid peroxide as an active ingredient is an effective concentration, which comprises three of the checkers according to claim 1, wherein the weight ratio of the potassium iodide and the thiosulfate ion is different between said checkers.

11. A method of determining whether or not a concentration of an organic carboxylic acid peroxide in an aqueous composition for sterilization comprising the organic carboxylic acid peroxide as an active ingredient is an effective concentration, by using a color-change reaction of a checker comprising potassium iodide, thiosulfate ion, and starch, wherein two or more of the checkers having a different weight ratio of the potassium iodide and the thiosulfate ion to each other are used in combination.
